(19) United States
(12) Patent Application Publication
(10) Pub. No.: US 2026/0083362 A1
(43) Pub. Date: Mar. 26, 2026

(54) ELECTRO-OPTICAL DEVICE, AN APPARATUS COMPRISING THE ELECTRO-OPTICAL DEVICE AND A METHOD OF CONDUCTING FUNCTIONAL NEAR-INFRARED SPECTROSCOPY ANALYSIS USING SUCH AN APPARATUS

(71) Applicant: The Chinese University of Hong Kong, New Territories (CN)

(72) Inventors: Sui-yin Agnes Chan, Tai Wai (CN); Xiaoran Ning, Tai Wai (CN)

(21) Appl. No.: 19/332,909

(22) Filed: Sep. 18, 2025

Related U.S. Application Data
(60) Provisional application No. 63/698,683, filed on Sep. 25, 2024.

(51) Int. Cl. A61B 5/1455; A61B 5/00; A61B 5/024

(52) U.S. Cl. CPC A61B 5/14552; A61B 5/002; A61B 5/0075; A61B 5/02433; A61B 5/14553; A61B 5/681

(57) ABSTRACT

An electro-optical device, an apparatus comprising the electro-optical device and a method of conducting functional near-infrared spectroscopy analysis using such an apparatus. The electro-optical device comprises: a light source arranged to irradiate a target spot of the head of a subject with light emission including a plurality of light components having distinct wavelengths, wherein light components with distinct wavelength are arranged to be partially absorbed by hemoglobin in blood passing though the target spot with distinct absorption ratios; and a light sensor arranged to detect light reflection from the target spot, wherein light reflection is adapted to be further processed for determination of relative concentrations of oxygenated hemoglobin and deoxygenated hemoglobin in the target spot based on a change of each of the light components with respective wavelength in the light reflection when compared to the light emission.

### TECHNICAL FIELD

This invention relates to an electro-optical device, an apparatus comprising the electro-optical device and a method of conducting functional near-infrared spectroscopy (fNIRS) analysis using such an apparatus. Particularly, although not exclusively, the invention relates to a fNIRS device incorporating multichannel sensors.

### BACKGROUND OF THE INVENTION

Dementia refers to an individual's decline in cognitive function to an extent that affects daily life and activities. Currently, dementia affects tens of millions of people worldwide, and it poses significant burden to individuals who suffer from it, their caregivers, and the society in general.

Dementia is generally progressive and may broadly include four stages: normal aging (normal cognition (NC)), subjective memory complaint (SMC), mild cognitive impairment (MCI), and dementia. Individuals with SMC usually have self-perceived or subjective cognitive decline. When compared with individuals without SMC, individuals with SMC have a higher risk of developing dementia. Further, when compared with individuals under normal aging, individuals with SMC may exhibit an increased risk of abnormalities in dementia-related biomarkers, regional brain hypometabolism, and/or atrophy in the medial temporal lobe. On the other hand, individuals with MCI exhibit lower performance on neuropsychological assessments but can maintain independent living abilities. MCI can be further categorized into amnestic MCI (aMCI), characterized by memory impairment, and non-amnestic MCI (naMCI), characterized by impairments in cognitive domains other than memory. aMCI may be more predictive of Alzheimer's disease (AD) whereas naMCI may be more predictive of other dementia subtypes.

Recent advancements in pharmacological interventions for early-stage dementia have highlighted the importance of identifying early signs of dementia before the final dementia stage is reached.

Conventionally, the diagnosis of MCI and dementia is based heavily on clinical diagnosis using standardized neuropsychological tests and clinical interviews. A problem associated with this approach is that it can be labor intensive.

To address this problem, more recently, the detection of the preclinical stage of dementia involves identifying abnormalities associated with related biomarkers. To date, the core biomarkers for dementia predominantly depend on positron emission tomography (PET) and the analysis of cerebrospinal fluid (CSF) and plasma samples. These indicators encompass alterations in A (amyloid beta) and T (tau) that can be identified through PET scans, which are useful for determining the transition/stage associated with dementia. However, a problem associated with this approach is that it is invasive (e.g., to obtain the samples).

### SUMMARY OF THE INVENTION

In accordance with a first aspect of the present invention, there is provided an electro-optical device for use in functional near-infrared spectroscopy (fNIRS), comprising: a light source arranged to irradiate a target spot of the head of a subject with light emission including a plurality of light components having distinct wavelengths, wherein light components with distinct wavelength are arranged to be partially absorbed by hemoglobin in blood passing though the target spot with distinct absorption ratios; and a light sensor arranged to detect light reflection from the target spot, wherein light reflection is adapted to be further processed for determination of relative concentrations of oxygenated hemoglobin and deoxygenated hemoglobin in the target spot based on a change of each of the light components with respective wavelength in the light reflection when compared to the light emission.

In accordance with the first aspect, the light emission includes four light components with wavelengths at 770 nm, 810 nm, 855 nm and 885 nm.

In accordance with the first aspect, the light source includes a plurality of light emitting diodes.

In accordance with the first aspect, the light sensor is adapted to detect light reflection with a dark count as low as a single photon.

In accordance with the first aspect, the light sensor includes silicon photomultipliers (SiPM) sensors.

In accordance with the first aspect, the electro-optical device comprises a plurality individual pairs of the light source and the light sensor, wherein each pair is arranged to provide a plurality of individual channels of light reflection representing cerebral blood dynamic and or hemoglobin change in a plurality of distinct target spots.

In accordance with a second aspect of the present invention, there is provided an apparatus for use in functional near-infrared spectroscopy (fNIRS), comprising: a fNIRS module comprising the optical device in accordance with the first aspect, wherein the plurality of individual pairs of the light source and the light sensor are arranged in an array; and a processing module arranged to process the light reflection sampled by the light sensor to analysis cerebral blood dynamic and/or hemoglobin change of a target area covering the plurality of distinct target spots of the head of the subject.

In accordance with the second aspect, the fNIRS module is provided on a wearable head-mount structure arranged to facilitate fixing positions of the light source and the sensors to a scalp surface or a skin surface of the head of the subject.

In accordance with the second aspect, the wearable head-mount structure includes a headband.

In accordance with the second aspect, the apparatus further comprises a fNIRS control module arranged to control of the fNIRS module.

In accordance with the second aspect, the apparatus further comprises at least one auxiliary functional module arranged to provide a function different from that provided by the fNIRS module.

In accordance with the second aspect, each of the at least one auxiliary functional module and the fNIRS module is individually powered.

In accordance with the second aspect, the auxiliary functional module includes a motion sensor arranged to measure a movement of the head of the subject.

In accordance with the second aspect, the motion sensor includes an inertial measurement unit provided on the wearable head-mount structure.

In accordance with the second aspect, the motion sensor includes a 3-axis accelerometer and a 3-axis gyroscope.

In accordance with the second aspect, the auxiliary functional module includes a pulse oximeter arranged to measure oxygen saturation level in blood and pulse rate of the subject.

In accordance with the second aspect, the pulse oximeter is provided separately from the separable head-mount structure.

In accordance with the second aspect, the pulse oximeter is provided on a wristband.

In accordance with the second aspect, the apparatus further comprises a central control module arranged to communicate with the fNIRS module, the processing module and the auxiliary functional module via a wireless communication link.

In accordance with the second aspect, the wireless communication link includes WiFi and/or Bluetooth.

In accordance with the second aspect, the processing module is provided in an external computer.

In accordance with the second aspect, the processing module includes a machine-learning based processing engine arranged to process the light reflection sampled by the light sensor to determine a physiological activity of the subject.

In accordance with the second aspect, the machine-learning based processing engine is further arranged to process supplementary physiological parameters sampled by the auxiliary functional module so as to isolate true hemoglobin changes from physiological noise embedded in the light reflection sampled by the light sensor.

In accordance with a third aspect of the present invention, there is provided a method of conducting functional near-infrared spectroscopy (fNIRS) analysis, comprising the steps of: mounting the wearable head-mount structure of the apparatus in accordance with the second aspect to the head of the subject; activating the fNIRS module to generate light reflection for being sampled by the light sensor; and processing the light reflection sampled by the light sensor to analysis cerebral blood dynamic and/or hemoglobin change of a target area covering the plurality of distinct target spots of the head of the subject.

In accordance with the third aspect, the method further comprises the step of providing supplementary physiological parameters sampled by at least one auxiliary functional module so as to isolate true hemoglobin changes from physiological noise embedded in the light reflection sampled by the light sensor.

In accordance with the third aspect, the step of providing supplementary physiological parameters sampled by the auxiliary functional module comprises the step of detecting movement of the head of the subject by a motion sensor.

In accordance with the third aspect, the motion sensor includes an inertial measure unit provided on the wearable head-mount structure.

In accordance with the third aspect, the wearable head-mount structure includes a headband.

In accordance with the third aspect, the step of providing supplementary physiological parameters sampled by the auxiliary functional module comprises the step of determining an oxygen saturation level (SpO₂) in blood and/or pulse rate of the subject by a pulse oximeter.

In accordance with the third aspect, the pulse oximeter is provided on a wristband.

### BRIEF DESCRIPTION OF THE DRAWINGS

Embodiments of the invention will now be described, by way of example, with reference to the accompanying drawings in which:

FIG. 1 is a schematic diagram illustrating an apparatus for functional near-infrared spectroscopy (fNIRS) in accordance with an embodiment of the present invention.

FIG. 2 is a flow diagram showing an example operation of conducting fNIRS analysis using fNIRS data obtained by the apparatus of FIG. 1.

FIG. 3 is a block diagram illustrating the apparatus for functional near-infrared spectroscopy (fNIRS) of FIG. 1.

FIG. 4 is a portion of the block diagram of FIG. 3 showing a fNIRS module in connection with a fNIRS control module in the apparatus of FIG. 1.

FIG. 5 is a block diagram showing operations of components in an electro-optical device of a fNIRS module of the apparatus of FIG. 1.

FIG. 6 is portion of the block diagram of FIG. 3 showing an IMU module and a Pulse oximeter module in the apparatus of FIG. 1.

FIG. 7 is a portion of the block diagram of FIG. 3 showing a computer and a central control module in the apparatus of FIG. 1.

FIG. 8 is schematic diagram illustrating positions of the fNIRS optodes and measurement channels in one embodiment of the invention;

FIG. 9 is a set of plots of HbO signal vs. time of all channels of measurement in an experiment of FIG. 8;

FIG. 10 is a set of plots of HbO signal vs. time of Channel 9 and 12 of all channels illustrated in FIG. 9.

### DETAILED DISCLOSURE OF THE INVENTION

The inventors, through their experiments and trials, devised that Current fNIRS devices on the market are affected, to varying degrees, by three main deficiencies (either all three or one or two of them): 1. Suboptimal Signal-to-Noise Ratio (SNR), 2. Significant artifacts caused by inconvenient wearability, excessive device weight, or obstructive connecting cables, and 3. Difficulty in correcting contaminated signals. Our device effectively addresses all three issues simultaneously. It employs higher precision optical detectors, utilizes a completely wireless configuration, and incorporates auxiliary signal sources for enhanced signal correction.

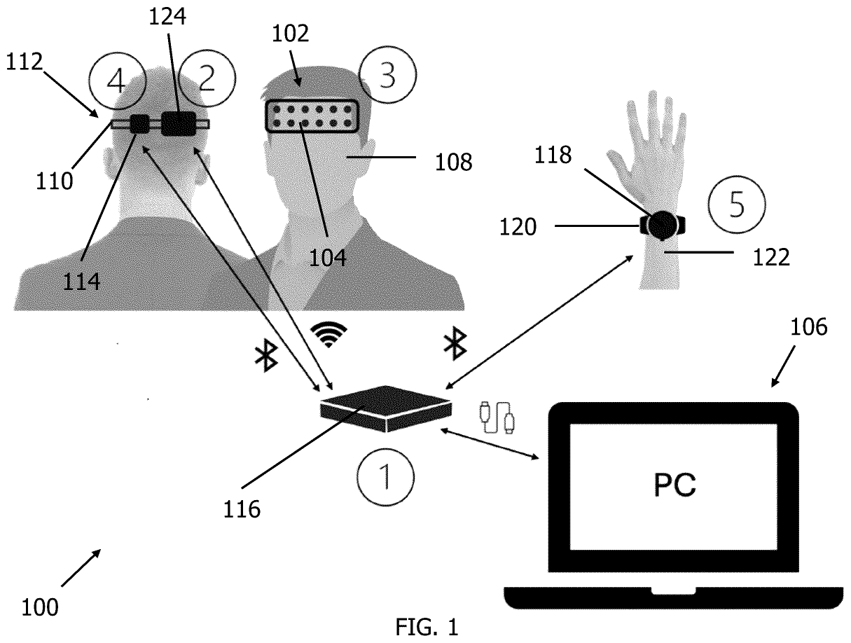

With reference to FIG. 1, there is shown an example embodiment of an apparatus 100 for use in functional near-infrared spectroscopy (fNIRS). The apparatus 100 comprises a fNIRS module 102 including an electro-optical device 104 and a processing module 106, where in the electro-optical device 104 comprises a light source arranged to irradiate a target spot of the head 108 of a subject with light emission including a plurality of light components having distinct wavelengths, wherein light components with distinct wavelength are arranged to be partially absorbed by hemoglobin in blood passing though the target spot with distinct absorption ratios; and a light sensor arranged to detect light reflection from the target spot, wherein light reflection is adapted to be further processed for determination of relative concentrations of oxygenated hemoglobin and deoxygenated hemoglobin in the target spot based on a change of each of the light components with respective wavelength in the light reflection when compared to the light emission. In addition, the plurality of individual pairs of the light source and the light sensor are arranged in an array in the apparatus 100; and the processing module 106 is arranged to process the light reflection sampled by the light sensor to analysis cerebral blood dynamic and/or hemoglobin change of a target area covering the plurality of distinct target spots of the head 108 of the subject.

In this embodiment, the apparatus 100 includes a plurality of individual parts or modules being fixed to different parts of the body of a subject, by a headband-like wearable head-mount structure 110, such as an electro-optical device 104 being mounted to a forehead of the subject for sampling reflection light signals at that target area covering multiple target spots irradiated by the light sources. As the light travels through the brain, it is absorbed and scattered by the tissue. Oxygenated hemoglobin (HbO) and deoxygenated hemoglobin (HbR) absorb light at different rates. For example, oxygenated hemoglobin absorbs more light at wavelengths above 790 nm, while deoxygenated hemoglobin absorbs more at wavelengths below this wavelength. Certain amount of light is reflected to the light sensor of the electro-optical device 104, thus by analyzing the differences in absorption in different wavelengths, fNIRS can estimate the concentrations of oxygenated hemoglobin and deoxygenated hemoglobin in the brain.

Referring to FIG. 1, the apparatus 100 is provided as a modular system, which includes a main fNIRS headband module 112 with 6 pairs of emitters detectors, forming in total of 16 channels. Another 2 sets of physiological monitoring modules providing auxiliary physiological information for improving analyzing fNIRS.

The same headband 110 may also fix other modules to the head 108 of the subject, including a motion sensor 114 arranged to measure a movement of the head 108 of the subject, where information related to the motion may be useful for correcting any data artifacts in the light signals sampled by the electro-optical device 104 on the same headband 110 caused by vigorous movements of the head 108 of the subject. Data, e.g. HbO/HbR data, sampled by the electro-optical device 104 and/or IMU data sampled by the motion sensor 114 may be further transmitted to a central control module 116 wirelessly, e.g. via WiFI or Bluetooth communication protocol.

In addition, the apparatus 100 comprises a pulse oximeter 118 being separately provided on other mounting structure, such as a wristband 120 for fixing the pulse oximeter 118 on a wrist 122 of the subject, for measure oxygen saturation level in blood and pulse rate of the subject. Similar to the motion sensor 114, the pulse oximeter 118 is an auxiliary functional module arranged to provide a function different from that provided by the fNIRS module 102 or the electro-optical device 104 for obtaining functional near-infrared spectroscopy related signals, and it may also provide supplementary physiological parameters for improving the fNIRS analysis. Similarly, data sampled by the pulse oximeter 118 may be transmitted to the central control module 116 wirelessly, and all physiological signal or data may be further processed by an external computer, which may be implemented as a processing module 106 for processing the light reflection sampled by the light sensor to analysis cerebral blood dynamic and/or hemoglobin change of a target area covering the plurality of distinct target spots of the head 108 of the subject.

The processing module 106 may calculate changes in hemoglobin concentration, which correlates with neural activity, e.g. increased blood flow to active brain regions (due to vasodilation) may indicate a higher neural activity or activity related to performing cognitive tasks.

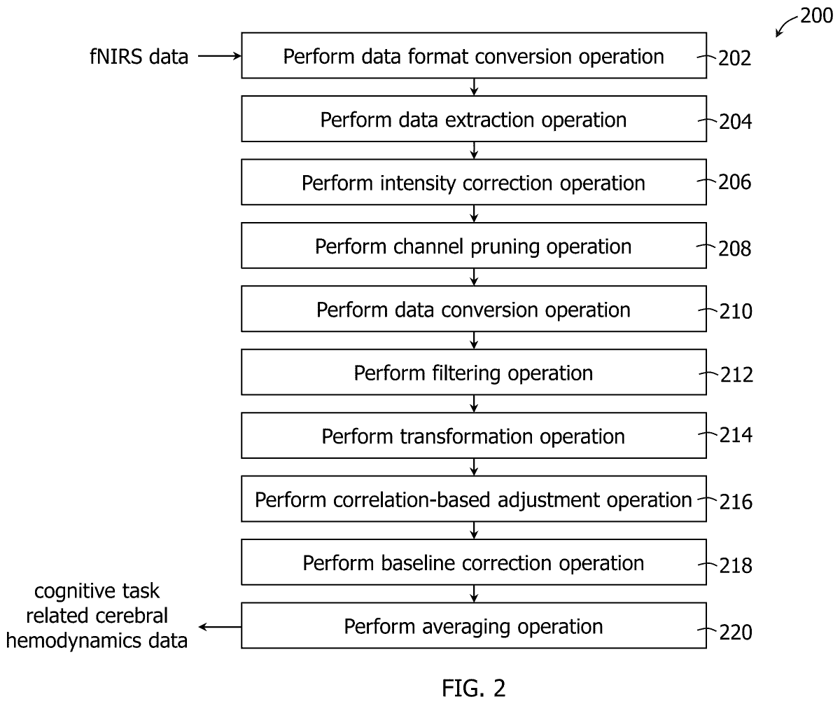

Referring also to FIG. 2, there is shown an example operation of fNIRS analysis conducted by using the apparatus 100 in accordance with embodiments of the present invention.

The functional units and modules of the electronic control circuit, such as the integer divider and the clock correction calculation module in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

The operation maybe facilitated by a computer system, such as the computer 106 referring to FIG. 1. The method 200 included operation 202, which includes performing a data format conversion operation. The data format conversion operation converts the fNIRS data from a first data format to a second data format different from the first data format. The second data format may be more suitable (e.g., faster, more secure, etc.) for subsequent processing than the first data format. In one example, operation 202 is not performed if it is determined that no data format conversion is required or that data format conversion is unsuitable.

Method 200 includes operation 204, performed after operation 202 (if operation 202 is performed), which includes performing a data extraction operation on the fNIRS data to obtain, at least, light intensity data, in particular raw light intensity data. In one example, the data extraction operation may extract, from the fNIRS data: parameters associated with source-detector geometry of the system, information associated with stimulus onsets of the cognitive task, information associated source-detector channels of the system, the (raw) light intensity data includes data time points and raw light intensity measurements of each of the channels of the system, and auxiliary signal.

Method 200 includes operation 206, which includes performing an intensity correction operation to remove all negative light intensity values from the light intensity data (obtained from operation 204). In one example, the intensity correction operation includes replacing each negative light intensity value with a respective distance value from 1.0 to a next integer double-precision number. In one example, the intensity correction operation includes, for each negative light intensity value, applying one or more increment signals, e.g., one or more dc signals, to all negative light intensity values in the light intensity data. In one example, operation 206 is not performed if it is determined that there are no negative light intensity values in the light intensity data.

Method 200 includes operation 208, which includes performing a channel pruning operation to prune one or more of the channels (e.g., disregard or remove the data associated with the one or more channels) based at least in part on one or more criteria. In one example, for each channel, the one or more criteria are associated with light intensity values obtained from the channel and one or more thresholds. In one example, for each channel, the one or more criteria are associated with: mean light intensity value of the light intensity values obtained from the channel, standard deviation of light intensity values obtained from the channel, one or more light intensity value thresholds, and a signal-to-noise ratio threshold.

Method 200 includes operation 210, which includes performing a data conversion operation on the light intensity data to obtain optical density data.

Method 200 includes operation 212, which includes performing a filtering operation to at least partly remove noise, in particular high-frequency noise, from the optical density data. The filtering operation may be performed using a filter, such as a bandpass filter or a low pass filter. In one example, the filter includes an n^{th} (e.g., 3^{rd}) order Butterworth low pass filter. In one example, operation 212 is not performed, e.g., if it is determined that the noise is at an acceptable level.

Method 200 includes operation 214, which includes performing a transformation operation on the optical density data to obtain HbO data containing information associated with relative HbO concentration changes and/or HbR data containing information associated with relative HbR concentration changes. In one example, performing the transformation operation includes processing the optical density data based at least in part on modified Beer-Lambert law. In one example, performing the transformation operation includes processing the optical density data based at least in part on absorption coefficient values and distance factors.

Method 200 includes operation 216, which includes performing a correlation-based adjustment operation. In one example, the correlation-based adjustment operation is performed on the HbO data and/or the HbR data to obtain correlation-adjusted HbO data and/or correlation-adjusted HbR data. In one example, the correlation-based adjustment operation includes processing the HbO data and/or the HbR data based at least in part on a correlation function arranged to effectuate a negative correlation between concentration changes of HbO and concentration changes of HbR. In one example, operation 216 is not performed.

Method 200 includes operation 218, which includes performing a baseline correction operation. In one example, the baseline correction operation is performed on the HbO data and/or the HbR data to obtain baseline-corrected HbO data and/or baseline-corrected HbR data. In one example, the baseline correction operation is performed on the correlation-adjusted HbO data and/or the correlation-adjusted HbR data to obtain modified HbO data and/or modified HbR data. In one example, the baseline correction operation includes: processing the HbO data and/or the HbR data for each trial based at least in part on data obtained during the control task. In one example, operation 218 may be performed prior to operation 216. In one example, operation 218 is not performed.

Method 200 includes operation 220, which includes performing an averaging operation to obtain the cognitive task related cerebral hemodynamics data. The averaging operation may include: averaging the modified HbO data and/or the modified HbR data for each trial based at least in part on data obtained during the visual memory span cognitive task to obtain averaged HbO data and/or averaged HbR data for each trial. The averaging operation may further include: averaging the averaged HbO data and/or averaged HbR data for each trial across trials with the same condition, to obtain cognitive task based HbO data and/or cognitive task based HbR data. The averaging operation may further include: averaging the cognitive task based HbO data and/or cognitive task based HbR data to across all channels that have not been pruned, to obtain the cognitive task related cerebral hemodynamics data.

It should be appreciated by a skilled person that the abovementioned operation is an example only, where various modifications can be made to the method 200 to provide other embodiments of the invention. For example, in some embodiments, one or more of the operations 202-220 can be omitted (i.e., the method may lack one or more of those operations). For example, in some embodiments, the order of the operations 202-220 is different than that illustrated in method 200. That is, in some embodiments, the operations 202-220 can be performed in a different order, as feasible, appropriate, and applicable.

The terminology used herein Is for the purpose of describing particular embodiments only and is not intended to be limiting to the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

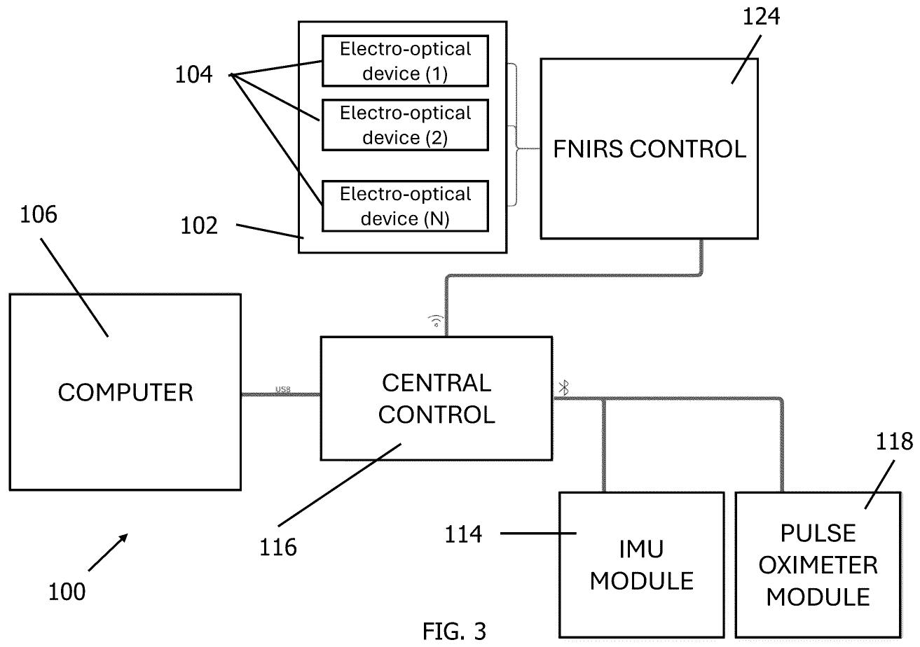

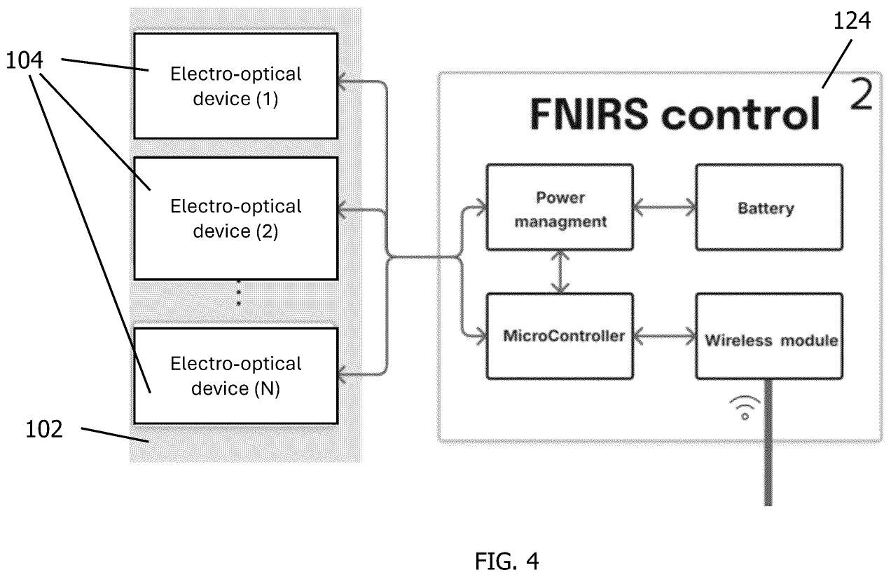

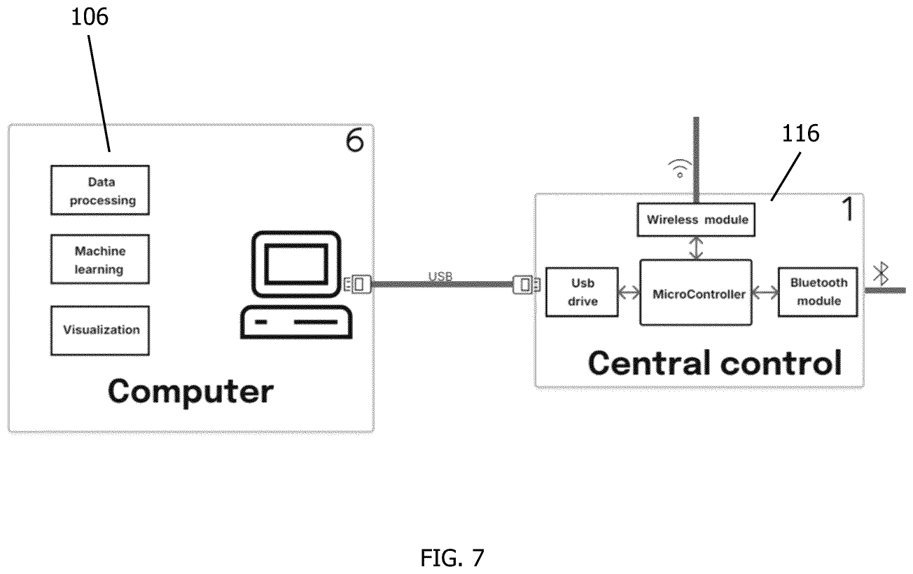

With reference to also to FIGS. 3 to 7, In accordance with the preferred embodiment of the present invention, there is provided an advanced wearable functional Near-Infrared Spectroscopy (fNIRS) device, incorporating several technological innovations to enhance performance and usability significantly.

Preferably, the light emission, generated by the electro-optical device 104 of the apparatus 100 for use in functional near-infrared spectroscopy (fNIRS), may include a plurality of light components emitting light with multiple wavelengths in the near-infrared spectrum, e.g. between 700 nm and 900 nm. More preferably, the device may employ four distinct wavelengths of LEDs as light sources. The four wavelengths include 770 nm, 810 nm, 855 nm and 885 nm, and the detected signal may be further processed to determined concentration change of oxyhemoglobin, deoxyhemoglobin by applying modified Beer-Lambert law (MBLL) for four wavelengths mentioned earlier.

Preferably, the light source includes a plurality of light emitting diodes each arranged to emit light with the selected peak wavelength mentioned earlier. Alternatively, other wavelengths or other types of light source such as laser diodes may also be used.

Advantageously, this multi-wavelength emission and detection pair allows for more precise monitoring and analysis of cerebral blood dynamics and hemoglobin changes, enhancing data accuracy and reliability.

Preferably, the light sensor includes silicon photomultipliers (SiPM) sensors, which may detect light reflection with a dark count as low as a single photon. Silicon Photomultiplier (SiPM) is a solid-state photodetector that is highly sensitive to light, capable of detecting single photons. It operates using an array of microcells, each containing a single-photon avalanche diode (SPAD) that works in Geiger mode. When a photon is absorbed, it triggers an avalanche of charge carriers, resulting in a measurable current pulse.

With its high sensitivity and low dark count of SiPM, signal detection capabilities in low-light conditions may be enhanced, thus signal-to-noise ratio may be substantially increased. As appreciated by a skilled person in the field, other types of high sensitivity light detector or photodetector may be used.

In a preferred embodiment, multiple LEDs and SiPM sensors are provided in a fNIRS module, in which the light source and the light sensor are arranged in a pair, and each pair is arranged to provide a plurality of individual channels of light reflection representing cerebral blood dynamic and or hemoglobin change in a plurality of distinct target spots. The individual pairs of light source and sensors may be preferably arranged in an array, such that it may be provided as a unitary module that can be fixed to a scalp surface or a skin surface of the head of the subject using a headband or other wearable head-mount structure. On the headband, a fNIRS control module 124 may be provided for control of the fNIRS module 102, i.e. operation of each the light sources and sensors, and the transmission of data collected by the sensors.

Preferably, the apparatus 100 further comprises at least one auxiliary functional module arranged to provide a function different from that provided by the fNIRS module 102. For example, the apparatus 100 may also comprise a motion sensor 114, such as an inertial measurement unit (IMU), arranged to measure a movement of the head 108 of the subject. The IMU may be provided on the same headband 110 or wearable head-mount structure having the fNIRS module 102 such that it may be worn by the subject.

Preferably, the IMU sensor may be a 6-Axis IMU-a device which measures and reports a body's specific force, angular rate, and optionally the magnetic field surrounding the body. In one example embodiment, the IMU may contain both a 3-axis accelerometer and a 3-axis gyroscope. In some alternative embodiments, single accelerometers/gyroscopes may be deployed to sample motions along different axes may be included in the IMU.

Advantageously, fNIRS may be useful in settings where movement is involved, such as studies with children or patients who may not be able to stay still for an fMRI scan. It's also more portable compared to other imaging techniques, thus it may be advantageous to capture and consider also the movement of the head of the subject for correcting or compensating any error induced by the movement of the head.

Additionally or optionally, the apparatus 100 includes a pulse oximeter 118 arranged to measure oxygen saturation level in blood and pulse rate of the subject. Pulse oximeter is a non-invasive medical device that measures the oxygen saturation level (SpO₂) in the blood and the pulse rate, and the SpO₂ parameter may be useful for a more precise estimation of the amount/concentration of HbO and HbR in blood. Preferably, the pulse oximeter 118 may be provided separately from the separable head-mount structure 110, such as on a wristband 122, similar to a smart watch. Alternatively, SpO₂ or other cardiovascular parameters may be sampled by other devices or modules and is further processed or considered in the fNIRS analysis.

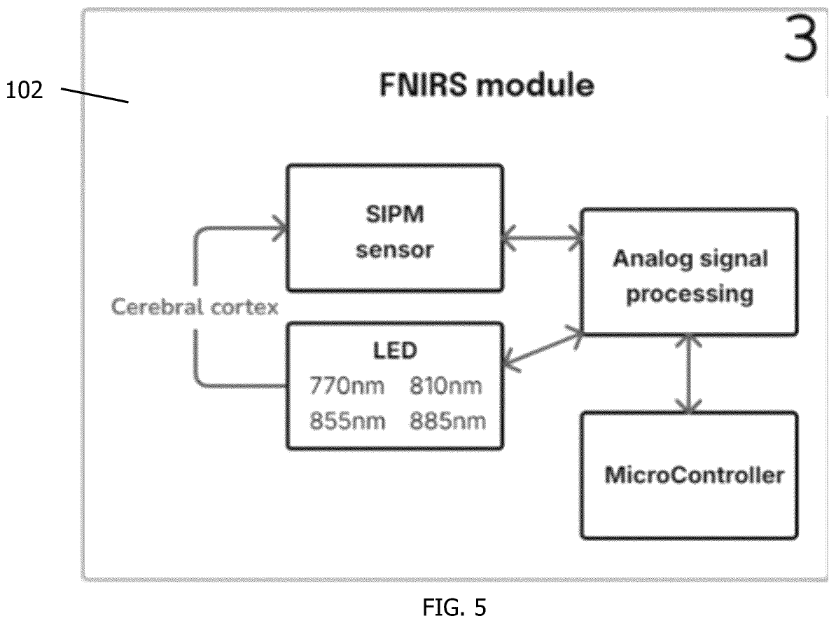

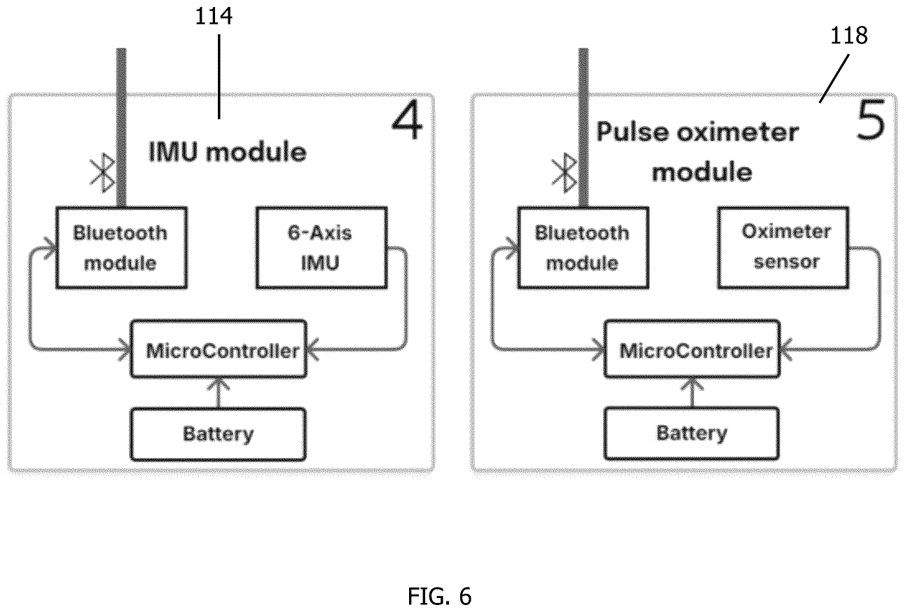

Preferably, the auxiliary functional modules and the fNIRS module are individually powered and controlled, such that each module supports plug-and-play functionality, usable independently or in conjunction. In addition, all sensor modules may communicate with a desktop central control unit via wireless protocols, which then connects to a personal computer a suitable communication link. For example, referring to FIGS. 5 and 6, each of the fNIRS module 102, the IMU module 114 and the pulse oximeter module 118 includes a dedicated microcontroller and a battery. Alternatively, one or more of the modules may share power from the same battery source.

Preferably, the apparatus 100 may further comprise a central control module 116 arranged to communicate with the fNIRS module 102, the processing module 106 and the auxiliary functional module via a wireless communication link, such as WiFi and/or Bluetooth communication link. Alternatively, wired communication link, such as USB may also be included. With the communication network provided, the processing module may be provided in an external computer.

The main fNIRS headband 112 uses WiFi to communicate with the central control unit 116 while other sensors communicate via Bluetooth. Advantageously, all sensor modules in the apparatus 100 communicate with a desktop central control unit 116 via wireless protocols, which in turn connects to a personal computer 106 via a USB cable, minimizing data errors caused by physical connections.

By integrating data from multiple physiological monitors and employing machine learning algorithms, the system can help identifies and mitigates artifacts caused by movement and poor coupling, so as to improve artifact mitigation and to optimize the fNIRS analysis. It may also help removing components unrelated to hemodynamic changes, further improving data quality and precision in analysis.

Preferably, the processing module 106 includes a machine-learning based processing engine arranged to process the light reflection sampled by the light sensor to determine a physiological activity of the subject. In addition, the machine-learning based processing engine may further process supplementary physiological parameters sampled by the auxiliary functional module so as to isolate true hemoglobin changes from physiological noise embedded in the light reflection sampled by the light sensor

The machine-learning based processing engine may be implemented as a processor within a computer server or the external computer which process the sampled data. In this embodiment, the system comprises a server which includes suitable components necessary to receive, store and execute appropriate computer instructions. The components may include a processing unit, including Central Processing Unit (CPUs), Math Co-Processing Unit (Math Processor), Graphic Processing Unit (GPUs) or Tensor processing united (TPUs) for tensor or multi-dimensional array calculations or manipulation operations, read-only memory (ROM), random access memory (RAM), and input/output devices such as disk drives, input devices such as an Ethernet port, a USB port, etc. Display such as a liquid crystal display, a light emitting display, or any other suitable display and communications links. The server may include instructions that may be included in ROM, RAM or disk drives and may be executed by the processing unit. There may be provided a plurality of communication links which may variously connect to one or more computing devices such as a server, personal computers, terminals, wireless or handheld computing devices, Internet of Things (IoT) devices, smart devices, edge computing devices, cloud devices. At least one of a plurality of communications links may be connected to an external computing network through a telephone line or other type of communications link.

The server may include storage devices such as a disk drive which may encompass solid state drives, hard disk drives, optical drives, magnetic tape drives or remote or cloud-based storage devices. The server may use a single disk drive or multiple disk drives, or a remote storage service. The server may also have a suitable operating system which resides on the disk drive or in the ROM of the server.

The computer or computing apparatus may also provide the necessary computational capabilities to operate or to interface with a machine learning network, such as neural networks, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time.

Facilitated by the modular system and machine learning enhancement, a functional Near-Infrared Spectroscopy (fNIRS) system featuring multiple plug-and-play physiological monitoring modules, including but not limited to an fNIRS headband module, optical heart rate monitor, blood pressure monitor, pulse oximeter, IMU sensor, and temperature sensor is provided. Each module can be used independently or in conjunction, enhancing the device's flexibility and multifunctionality.

The system may further include structures and interfaces that enable the application of machine learning algorithms for automated optimization of signal processing, efficiency enhancement, and precision improvement. These machine-learning based processing engine is specifically used to identify and reduce artifacts in real-time caused by environmental changes or improper user operation, and to enhance the integration and analysis of fNIRS data with other physiological data.

These embodiments may be advantageous in that, an enhanced wearable functional Near-Infrared Spectroscopy (fNIRS) device is provided, which may address several limitations present in current technologies.

Advantageously, enhanced Signal-to-Noise Ratio (SNR) may be achieved, by employing advanced sensor technologies and signal processing algorithms, which significantly improves the SNR, to provide more accurate and reliable data for the monitoring and diagnosis of pathological conditions such as dementia. A functional Near-Infrared Spectroscopy (fNIRS) device characterized by employing four distinct wavelengths of LEDs as light sources may also provide more precise monitoring of cerebral blood flow and hemoglobin changes compared to dual-wavelength sources.

Moreover, better SNR may also be achieved by employing Silicon Photomultipliers (SiPM) Sensors, which may enhance signal detection capabilities in low-light environments and improve the signal-to-noise ratio, due to SiPM's high sensitivity and low dark count characteristics.

In addition, the improved apparatus in accordance with embodiments of the prevent invention allows effective differentiation of physiological signals, by integrating multi-source signal processing techniques and additional physiological monitoring modules, the system may more effectively isolate true hemoglobin changes from complex physiological noise, enhancing the efficacy and precision of experiments.

Furthermore, by implementing a comprehensive wireless transmission protocol, a fully wireless transmission protocol where various types of sensor modules communicate via wireless means with a central control module placed on a desktop may be utilized. Wearable devices with wireless modules may connect to external computers for centralized data processing and analysis. This entirely wireless setup markedly reduces the dependency on physical data cables and the overall weight of the device, substantially improving portability and practicality while also minimizing the potential for human error during everyday use.

The invention may be used in clinical medical applications, in which neurologists may use the device to monitor and assess brain disorders such as dementia and Parkinson's disease, particularly for diagnosing conditions and tracking treatment efficacy. Rehabilitation Centers employ the device to evaluate the progress of patients recovering from brain injuries or strokes, monitoring changes in brain function during treatment.

Universities and Research Institutions can utilize this device to conduct real-time monitoring of behavior and brain functions in naturalistic settings for academic researches. The invention supports a wide range of studies from biomedical engineering to behavioral science.

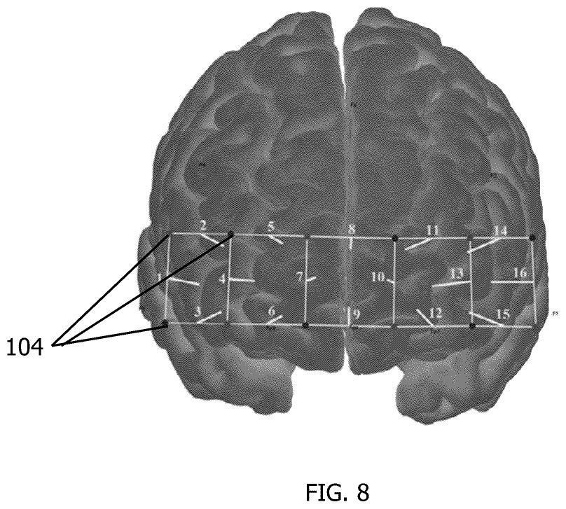

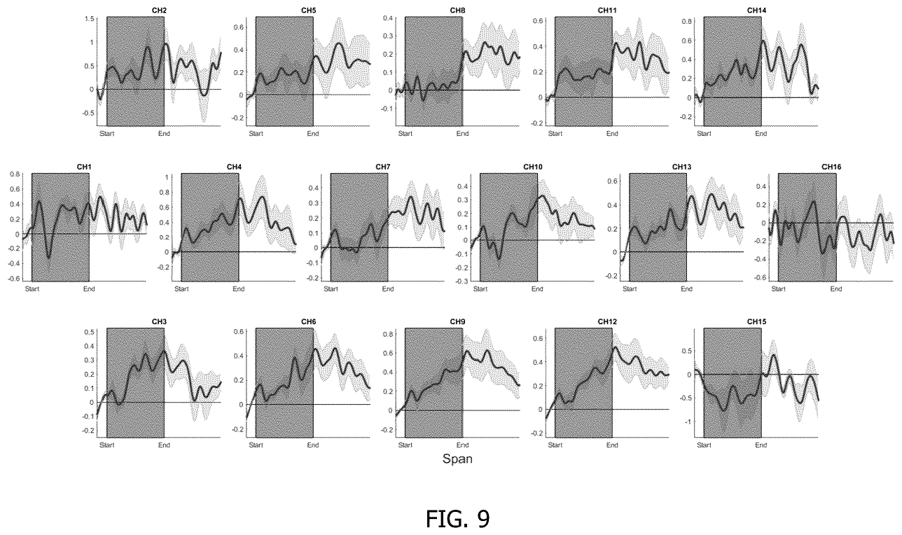

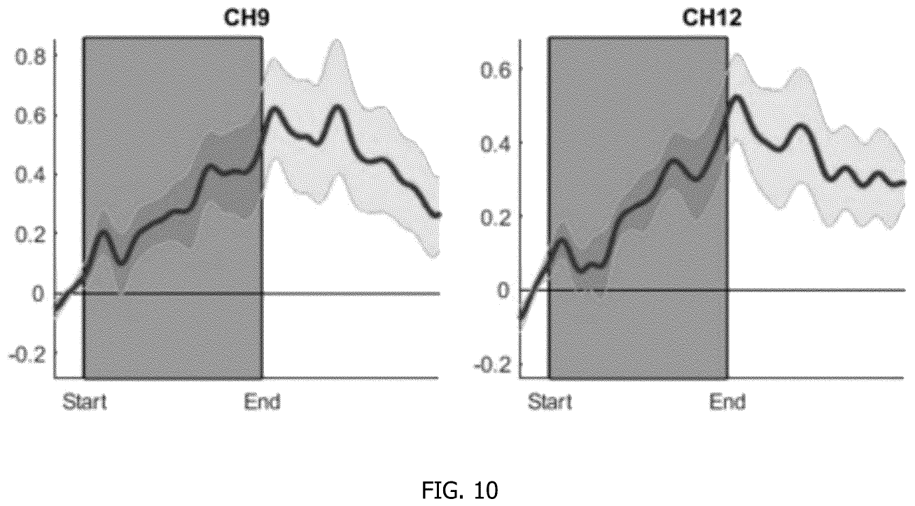

With reference to FIG. 8, there is shown example positions of the light source and sensor pairs, also known as fNIRS optodes, and measurement channels in an example experiment conducted by the inventors. The example positions of the fNIRS optodes and measurement channels may be used to obtain the fNIRS data. In this example, to measure prefrontal hemodynamic activity during a category fluency (CF) task, the apparatus in accordance with embodiments of the present invention was used to estimate the relative concentration of HbO in the participants' prefrontal cortex, using the modified Beer-Lambert Law. As shown in FIG. 6, the fNIRS system in this example includes six sources and six detectors arranged in a 2×6, and a total number of 16 channels are monitored, with the experimental results, in particular measured HbO, illustrated referring to FIGS. 9 and 10.

Experiment parameters are listed as follows:
n=8 (6F2M)
Preprocessing
Intensity to OD
Low-pass filter (0.1 Hz)
OD to concentration (ppf=6.0 for both wavelengths)
Correlation based signal improvement for motion correction
Block average

It is observed that there is an increase in HbO when the participants are performing CF. According to FIGS. 9 and 10, increased HbO is observed, particularly in channels 2-6, 9, and 11-14, covered in the grey region under the graphs.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components, and data files assisting in the performance of specific functions, the skilled person will understand that the functionality of the software application may be distributed across a number of routines, objects, or components to achieve the same functionality desired herein.

It will also be appreciated that where the methods and systems of the present invention are either wholly implemented by computing systems or partly implemented by computing systems then any appropriate computing system architecture may be utilized. This will include stand-alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. An electro-optical device for use in functional near-infrared spectroscopy (fNIRS), comprising:
a light source arranged to irradiate a target spot of the head of a subject with light emission including a plurality of light components having distinct wavelengths, wherein light components with distinct wavelength are arranged to be partially absorbed by hemoglobin in blood passing though the target spot with distinct absorption ratios; and
a light sensor arranged to detect light reflection from the target spot,
wherein light reflection is adapted to be further processed for determination of relative concentrations of oxygenated hemoglobin and deoxygenated hemoglobin in the target spot based on a change of each of the light components with respective wavelength in the light reflection when compared to the light emission.

2. The electro-optical device in accordance with claim 1, wherein the light emission includes four light components with wavelengths at 770 nm, 810 nm, 855 nm and 885 nm.

3. The electro-optical device in accordance with claim 2, wherein the light source includes a plurality of light emitting diodes.

4. The electro-optical device in accordance with claim 1, wherein the light sensor is adapted to detect light reflection with a dark count as low as a single photon.

5. The electro-optical device in accordance with claim 4, wherein the light sensor includes silicon photomultipliers (SiPM) sensors.

6. The electro-optical device in accordance with claim 1, comprising a plurality individual pairs of the light source and the light sensor, wherein each pair is arranged to provide a plurality of individual channels of light reflection representing cerebral blood dynamic and or hemoglobin change in a plurality of distinct target spots.

7. An apparatus for use in functional near-infrared spectroscopy (fNIRS), comprising:
a fNIRS module comprising the electro-optical device in accordance with claim 6, wherein the plurality of individual pairs of the light source and the light sensor are arranged in an array; and
a processing module arranged to process the light reflection sampled by the light sensor to analysis cerebral blood dynamic and/or hemoglobin change of a target area covering the plurality of distinct target spots of the head of the subject.

8. The apparatus in accordance with claim 7, wherein the fNIRS module is provided on a wearable head-mount structure arranged to facilitate fixing positions of the light source and the sensors to a scalp surface or a skin surface of the head of the subject.

9. The apparatus in accordance with claim 8, wherein the wearable head-mount structure includes a headband.

10. The apparatus in accordance with claim 9, further comprising a fNIRS control module arranged to control of the fNIRS module.

11. The apparatus in accordance with claim 10, further comprising at least one auxiliary functional module arranged to provide a function different from that provided by the fNIRS module.

12. The apparatus in accordance with claim 11, wherein each of the at least one auxiliary functional module and the fNIRS module is individually powered.

13. The apparatus in accordance with claim 12, wherein the auxiliary functional module includes a motion sensor arranged to measure a movement of the head of the subject.

14. The apparatus in accordance with claim 13, wherein the motion sensor includes an inertial measurement unit provided on the wearable head-mount structure.

15. The apparatus in accordance with claim 14, wherein the motion sensor includes a 3-axis accelerometer and a 3-axis gyroscope.

16. The apparatus in accordance with claim 15, wherein the auxiliary functional module includes a pulse oximeter arranged to measure oxygen saturation level in blood and pulse rate of the subject.

17. The apparatus in accordance with claim 16, wherein the pulse oximeter is provided separately from the separable head-mount structure.

18. The apparatus in accordance with claim 17, wherein the pulse oximeter is provided on a wristband.

19. The apparatus in accordance with claim 11, further comprising a central control module arranged to communicate with the fNIRS module, the processing module and the auxiliary functional module via a wireless communication link.

20. The apparatus in accordance with claim 19, wherein the wireless communication link includes WiFi and/or Bluetooth.

21. The apparatus in accordance with claim 11, wherein the processing module is provided in an external computer.

22. The apparatus in accordance with claim 21, wherein the processing module includes a machine-learning based processing engine arranged to process the light reflection sampled by the light sensor to determine a physiological activity of the subject.

23. The apparatus in accordance with claim 22, wherein the machine-learning based processing engine is further arranged to process supplementary physiological parameters sampled by the auxiliary functional module so as to isolate true hemoglobin changes from physiological noise embedded in the light reflection sampled by the light sensor.

24. A method of conducting functional near-infrared spectroscopy (fNIRS) analysis, comprising the steps of:
mounting the wearable head-mount structure of the apparatus in accordance with claim 8 to the head of the subject;
activating the fNIRS module to generate light reflection for being sampled by the light sensor; and
processing the light reflection sampled by the light sensor to analysis cerebral blood dynamic and/or hemoglobin change of a target area covering the plurality of distinct target spots of the head of the subject.

25. The method in accordance with claim 24, further comprising the step of providing supplementary physiological parameters sampled by at least one auxiliary functional module so as to isolate true hemoglobin changes from physiological noise embedded in the light reflection sampled by the light sensor.

26. The method in accordance with claim 25, wherein the step of providing supplementary physiological parameters sampled by the auxiliary functional module comprises the step of detecting movement of the head of the subject by a motion sensor.

27. The method in accordance with claim 26, wherein the motion sensor includes an inertial measure unit provided on the wearable head-mount structure.

28. The method in accordance with claim 27, wherein the wearable head-mount structure includes a headband.

29. The method in accordance with claim 25, wherein the step of providing supplementary physiological parameters sampled by the auxiliary functional module comprises the step of determining an oxygen saturation level in blood and/or pulse rate of the subject by a pulse oximeter.

30. The method in accordance with claim 29, wherein the pulse oximeter is provided on a wristband.
